(19) United States
(12) Patent Application Publication
(10) Pub. No.: US 2007/0225596 A1
(43) Pub. Date: Sep. 27, 2007

(54) Implant, Apparatus and Method for Tracking a Target Area

(75) Inventors: Roman Iustin, Molndal (SE); Sten Nilsson, Uppsala (SE); Bengt Rosengren, Hovas (SE); Johan Linder, Goteborg (SE); Erik Isberg, Ellos (SE); Tomas Gustafsson, Molndal (SE); Bo Lennernas, Uddevalla (SE); Seymour Levitt, Edina, MN (US)

Correspondence Address: BIRCH STEWART KOLASCH & BIRCH, PO BOX 747, FALLS CHURCH, VA, 22040-0747, US

(73) Assignee: Micropos Medical AB, Goteborg (SE)

(21) Appl. No.: 11/578,704

(22) PCT Filed: May 3, 2005
(86) PCT No.: PCT/SE05/00646

§ 371 Date: May 25, 2007

(30) Foreign Application Priority Data
May 3, 2004 (SE) 0401148-2
Feb. 22, 2005 (SE) 0500410-6

(51) Int. Cl. A61B 5/05

(52) U.S. Cl. 600/424; 600/407

(57) ABSTRACT

The present invention relates to an implant (115) fixable relative to a target area (117) within a living body (105), comprising a transmitter (7; 107; 207; 307) arranged to emit an electromagnetic signal, wherein said electromagnetic signal is adapted to propagate with a wavelength 2 in said living body (105) so that a phase difference of said electromagnetic signal in at least three positions, preferably four, separated by a known distance (b) is detectable by a receiver (9; 109; 209; 309) for tracking variations of a position (p) of the implant (115) relative to said receiver (9; 109; 209; 309). Further, the present invention relates to an apparatus (1; 101; 201; 301), a system and a method for tracking a position (p) of a transmitter (7; 107; 207; 307). Additionally, the invention relates to the use of such a method for treatment of a target area (117) in a living body (105) by means of a radiation therapy treatment arrangement (100).

### TECHNICAL FIELD OF INVENTION

The present invention relates to an implant fixable relative to a target area within a living body, for locating and tracking said target area. Further, the present invention relates to an apparatus and a method for tracking a position of a transmitter in a lossy medium relatively to a receiver.

### BACKGROUND OF THE INVENTION

Patients with e.g. a cancer tumor are often treated with radiotherapy. Such a treatment is usually carried out in several fractions, typical 35 fractions during a period of 7 weeks. The plurality of fractions which are used serves to reduce the risk of severe side effects that is caused by the treatment.

In order to increase the precision of the treatment, the patient is examined with one or several pre-treatment examinations, such as X-rays, computer tomography (CT-scan), magnetic resonance imaging (MRI), gamma camera, positron emitting tomography (PET) or the like. In these examinations, the target area (e.g. a tumor), as well as healthy organs are outlined in order to optimize the treatment by concentrating the administrated dose to the target area and in the outmost possible extent avoid administration to the healthy organs.

However, due to organ and patient motions during the treatment, as well as between the treatments, resulting from respiration, blood flow, gastric motions, and other causes, it is required to add a specific margin around the target area in which the radiotherapy energy should be projected, in order to keep a good treatment efficiency. This requirement may cause that the healthy organs around the target will be affected by high energy radiation, thus increasing the risk for side effects as a result.

In order to reduce the risk for side effects during radiotherapy, or any other examination with a predefined anatomical structure (such as pre-treatment examinations mentioned above), different kind of target area positioning devices are used within the prior art. For instance, stereotactic frames, vacuum bags, laser with skin markers, fixation masks, X-rays with or without markers in the target area etc. may be used. These target area positioning devices are not intended to be used during the treatment process. Hence, the use of such pre-positioning systems give a precision during the treatment of approximately 2 cm around the tumor due to organ and patient motions, which may give a high risk for side effects. Thus, these pre-positioning systems still do not reduce the risks of side effects under accepted limits.

WO02/100485 discloses a system and a method for locating and tracking the position of a target area, such as a tumor or the like, within a body. The system includes one or more excitable beacons positioned in or near the target area, an external excitation source that remotely excites the beacons to produce an identifiable signal and a plurality of sensors spaced apart in a known geometry relative to each other. Further, a computer is coupled to the sensors and configured to use the beacon measurements to compare the position of the target area with an location of a machine iso-center of a radiotherapy arrangement. The computer also controls the movement of the patient and a patient support device so that the target area iso-center is coincident with the iso-center of the radiotherapy arrangement before and during the treatment.

The described system and method determine the position of the target area by measuring the direction and amplitude of the radio signal generated by the beacon.

WO03/011394 discloses an alternative system and process for monitoring of a targeted area of a patients body to locate the position of the targeted area in real time.

### SUMMARY OF THE INVENTION

An object of the invention is to improve the resolution for tracking of a target area located in a lossy medium.

Another object of the invention is to eliminate the need of high frequencies for tracking of a target area within a lossy medium.

Yet another object of the present invention is to eliminate the need of high frequencies, for tracking of a target area in a living body, that may be harmful for tissues and/or organs within the living body.

These and other objects, which will become apparent in the following description, are achieved by means of an implant, an apparatus and a method having the features defined in the appended claims.

A first aspect of the invention relates to an implant fixable relative to a target area within a living body, comprising a transmitter arranged to emit an electromagnetic signal, wherein said electromagnetic signal is adapted to propagate with a wavelength in said living body so that a phase difference of said electromagnetic signal in at least three positions, preferably four, separated by a known distance is detectable by a receiver for tracking variations of a position of the implant relative to said receiver, wherein said wavelength is selected so that a distance from the transmitter to each of said at least three positions is within the same integer number of wavelengths of the electromagnetic signal.

It is realized that the location of the receiver, and hence the location of the at least three positions where the electromagnetic signal from the transmitter is detected, is known. It is obvious for a person skilled in the art that the location of the receiver in space may be determined in different ways, for instance by a fix point, by optical measurements, etc.

An particular advantage of the implant according to the invention is that the emitted wavelength in the lossy medium may be greater than an intended implant motion relatively to said receiver. Thereby, the variations of position between the implant and the receiver may be kept within the length of one complete wavelength in the lossy medium, wherein the tracking of the implant may be performed in a preferred way by measuring the phase difference.

An additional advantage of the implant according to the invention is that the emitted electromagnetic signal from said transmitter may has a frequency which is able to propagate, e.g. through the tissues of a living body, with relatively low attenuation. Thereby said electromagnetic signal, intended to preferably be detected and measured by a phase detector, has a measurable signal intensity at the same time as a transmission through the living body which is substantially harmless.

The transmitter may be arranged to emit an electromagnetic signal adapted to propagate with a frequency within the range of 5-1000 MHz.

According to one embodiment the transmitter is arranged to emit an electromagnetic signal adapted to propagate with a frequency within the range of 5-900 MHz.

According to another embodiment the transmitter is arranged to emit an electromagnetic signal adapted to propagate with a frequency within the range of 5-450 MHz.

According to yet another embodiment the transmitter is arranged to emit an electromagnetic signal adapted to propagate with a frequency within the range of 5-350 MHz.

Preferably, said electromagnetic signal is adapted to propagate with a frequency within the range of 5-200 MHz, and in particular within the range of 5-100 MHz.

A second aspect of the invention relates to an implant fixable relative to a target area within a living body, comprising a transmitter arranged to emit an electromagnetic signal adapted to propagate with a frequency, wherein said frequency is within the range of 5-1000 MHz, preferably 5-900 MHz, more preferably 5-450 MHz and especially 5-350 MHz, so that a phase difference of said electromagnetic signal in at least three positions, preferably four, separated by a known distance is detectable by a receiver for tracking variations of a position of the implant relatively to a receiver.

It shall be noted that all alternative embodiments according to the first aspect of the invention is applicable to the second aspect of the invention, and vice versa.

Preferably, a wavelength of the electromagnetic signal propagating in said lossy medium is greater than the distance between the implant and the receiver.

An advantage by the use of a wavelength greater than the distance between the implant and the receiver, is that the motion of the implant is allowed to vary within a large range without limiting the measurements of the position between the implant and the receiver reference point.

According to one embodiment, the transmitter is arranged to be energized by an external excitation source located outside the living body. The external excitation source may be energizing the implant either wire less or via a wire. According to an especially preferred embodiment of the invention, the transmitter comprises a frequency converter. The use of an external excitation source and an implant comprising a frequency converter makes it possible to provide a wireless implant which may be the more comfortable for a person wearing the implant.

The frequency converter may either be a frequency mixer, a frequency divider or a frequency multiplier. Depending on the type of converter, an energizing signal from the excitation source is selected such as the implant generates a suitably electromagnetic signal in accordance to the abovementioned.

Preferably, in this case, the implant comprises a mixer circuit for receiving and mixing a first energizing signal with a first frequency and a second energizing signal with a second frequency for generation of said emitted electromagnetic signal, the frequency of said emitted electromagnetic signal substantially corresponding to the difference between said first and second frequencies. The frequency of said emitted electromagnetic signal emitted from the implant is further on referred to as a differentially frequency.

Further, the use of two signals for the energizing of the transmitter comprising said mixer circuit, gives the possibility to mix said frequencies for generation of an electromagnetic signal with the differentially frequency to be emitted from the implant. The advantage by using the differentially frequency is that the emitted signal from the implant may has a frequency that propagates in the tissues of the living body with relatively low attenuation compared to a signal with a considerably higher frequency, for instance in the field of microwaves. Hence, the low attenuation and the low frequency enables the measurement of the phase difference, with a sufficient signal intensity kept at the receiver.

The use of an energizing signal with a high frequency is preferred, since the selectivity of the transmitter is suitable achieved in order to select the received and transmitted frequency.

Further the length of the implant preferably is between 5-40 mm and especially between 15-25 mm. In an especially preferred embodiment the implant has a diameter of about 3mm.

According to an alternative embodiment, the transmitter of the implant is energized via a wire. In this case, the implant preferably is connected to a wave generator located outside the living body, wherein the frequency of the emitted signal from the implant preferably is selected by adjusting the wave generator.

According to a further embodiment, the implant includes a source of energy for energizing of the transmitter. In this alternative embodiment, the implant preferably comprises a wave generator for generation of the signal to be emitted from the implant, wherein the frequency preferably is predetermined by way of the wave generator design. Advantageously, the implant is initially activated before the treatment, for instance by the way of a trigger signal from the outside of the living body, and inactivated after the treatment is finished. Activation only during the treatment is preferably used for saving energy.

A third aspect of the present invention relates to an apparatus for tracking a position of a transmitter located in a lossy medium, said transmitter being adapted to emit an electromagnetic signal that propagates in the lossy medium, comprising a receiver for detecting and measuring a phase difference of said electromagnetic signal in at least three positions, preferably four, separated by a known distance, for tracking the position of the transmitter relative to said receiver.

A fourth aspect of the invention relates to an apparatus for tracking a position of a transmitter located in a lossy medium, said transmitter being adapted to emit an electromagnetic signal that propagates in the lossy medium, comprising a receiver for detecting and measuring a phase difference of said electromagnetic signal in at least three positions, preferably four, separated by a known distance, for tracking the position of the transmitter relative to said receiver, wherein the at least three positions are located so that a distance from the transmitter to each one of said at least three positions is within the same integer number of wavelengths of the electromagnetic signal propagating in the lossy medium.

The receiver may be arranged to detect and measure said electromagnetic signal emitted from the transmitter with a frequency within the range of 5-1000 MHz, preferably 5-900 MHz, more preferably 5-450 MHz and especially 5-350 MHz.

It is to be noted that such a lossy medium may be constituted by salt water, tissues of a living body or the like. By the term lossy medium means, a medium in which the amplitude of the signal decreases by a factor of e⁻¹ at a distance called the skin depth or the depth of penetration. The apparatus according to the invention may be used for different applications, such as tracking of a target area in a living body, tracking of divers or submarine robots position in water, etc.

By measuring the phase difference of the electromagnetic signal from the transmitter in at least three positions, the position of the transmitter may be determined without knowledge of the phase generated at the transmitter, by way of comparing the signal received at the at least three positions.

It shall be noted that all alternative embodiments according to the third aspect of the invention is applicable to the fourth aspect of the invention, and vice versa.

According to other embodiments of the apparatus, the receiver may be arranged to detect and measure said electromagnetic signal within the different ranges of emitted frequencies given for the transmitter of the implant described above.

According to one embodiment of the apparatus, one of said at least three positions for detecting and measuring a phase difference of said electromagnetic signal may be arranged as a reference, wherein a phase difference, for the at each position detected electromagnetic signal from the transmitter, is determined between the reference and each one of the other positions. Using one of the at least three positions as a reference makes it possible to track the position of the transmitter by means of a phase detector without use of a reference signal provided from the transmitter. This is especially an advantage when the transmitter located within the lossy medium is wireless.

The apparatus can further comprise an excitation source to be located outside the lossy medium, for energizing said transmitter, e.g. as was described in relation to the first and second aspect of the invention.

In the case where the transmitter is adapted to receive two different energizing signals, the excitation source can comprise two antennas arranged for emitting a first and a second energizing signal respectively. The antennas are preferably fed by a wave generator for the generation of the energizing signals with a slightly difference in frequencies. It is obvious for a person skilled in the art that the energizing signals may be achieved in several ways, wherein the abovementioned is one example. Further, it is obvious for a person skilled in the art that such an wireless excitation source, generating at least two energizing signals with a slightly difference in frequencies, as well can be located within the lossy medium.

It shall be understood that only one antenna is needed for the wireless excitation of the transmitter comprising a frequency divider or a frequency multiplier.

The receiver preferably comprises an array of at least four sensors with a known distance between adjacent sensors. By arranging the sensors with a known distance, the measured phase difference of the electromagnetic signal emitted from the transmitter may be compared for determination of the transmitter position relatively to the receiver. The use of four sensors makes it possible to measure the relation between said receiver and the wireless transmitter in three dimensions, wherein the position of the transmitter, relatively to the receiver, may be monitored in real time. The determination of the transmitter position can be made by way of triangulation, neural networks etc.

It is obvious for a person skilled in the art that the use of an array comprising three sensors may give the possibility of monitoring the position of the wireless transmitter, relatively to the receiver, in two dimensions, for such an apparatus. Further, it shall be noted that an array comprising more than four sensors can be used for improvement of the accuracy of the measurements.

The distance between adjacent sensors is preferably shorter than half a wavelength of the detected electromagnetic signal propagating in the lossy medium, in order to keep the measured phase difference between the adjacent sensors below π radians.

A fifth aspect of the invention relates to a system for tracking a target area, comprising a transmitter fixable relative to said target area and adapted to emit a signal propagating in the lossy medium and an apparatus according to the third or fourth aspect of the invention. Further the transmitter may be provided as part of an implant according to the first or second aspect of the invention.

A sixth aspect of the invention relates to a method for tracking a position of a transmitter located in a lossy medium, comprising emitting an electromagnetic signal from the transmitter, detecting said electromagnetic signal in at least three positions, preferably four, separated by a known distance, determining a phase difference of said signal in said positions, and tracking the position of the transmitter relatively to a receiver based on said phase difference and said known distance.

A seventh aspect of the invention relates to a method for tracking a position of a transmitter located in a lossy medium, comprising emitting an electromagnetic signal from the transmitter, detecting said electromagnetic signal in at least three positions, preferably four, located so that a distance from the transmitter to each one of said at least three positions is within the same integer number of wavelengths of the electromagnetic signal propagating in the lossy medium, wherein said at least three positions are separated by a known distance, determining a phase difference of said signal in said positions, and tracking the position of the transmitter relative to a receiver based on said phase difference and said known distance.

The frequency of said electromagnetic signal may be within the range of 5-1000 MHz, preferably 5-900 MHz, more preferably 5-450 MHz and especially 5-350 MHz.

According to alternative embodiments of the method, the frequency emitted from the transmitter may be within the same ranges of frequencies as given for the transmitter of the implant described above.

It shall be noted that all alternative embodiments according to the sixth aspect of the invention is applicable to the seventh aspect of the invention, and vice versa.

According to one embodiment, said at least three positions may be located so that a distance from the transmitter to each one of said at least three positions is shorter than a wavelength of the electromagnetic signal propagating in the lossy medium.

According to one preferred embodiment, said method further comprises, energizing the transmitter by means of an excitation source located outside the lossy medium, for generation of said electromagnetic signal. This step of energizing the transmitter can be performed as mentioned above.

The method according to the invention, can be used when treating a target area in a living body by means of a radiation therapy treatment arrangement.

Additionally to the abovementioned, the shape of the wave front of the electromagnetic signal emitted from the transmitter may be measured in order to determine a distance between the transmitter and the receiver. The information regarding said distance may be used in combination with the abovementioned measurements of the phase difference. It shall be noted that the measurements of the shape of the wave front is an advantageously result from using the near field regions of the transmitter and the receiver.

In the vicinity of the implant and the outside antenna system, there often are permanent movable objects, such as the patient or the operator. Hence, the capacitance between each of the antennas and the ground may be permanently under changes, wherein the antennas may be sensitive to all movements in their vicinity and thus may leads to a malfunction. For reduction of this effect, the sensors, or sensor antennas, may be balanced with respect to the ground. According to one alternative embodiment, this is achieved by way of forcing the capacitance between the sensor antennas and the ground to be identical over the time. This can be established by using sensors arranged as electrostatically shielded antennas. This is especially suitable in medical applications, such as medical implantable communication systems, that contain an implantable transmitter in a lossy medium and an antenna system outside the lossy medium communicating with the transmitter.

### BRIEF DESCRIPTION OF THE DRAWINGS

By way of examples, embodiments of the invention will now be described with reference to the accompanying drawings in which

FIG. 1 shows a schematic perspective view of one embodiment of an apparatus for tracking a target area;

FIG. 2 shows a block diagram of a method for tracking a target area;

FIG. 3 shows a schematic side view of a radiation therapy treatment arrangement, comprising an apparatus and an implant for tracking a target area in a living body;

FIG. 4 shows a schematic perspective view of an alternative embodiment of an apparatus according to FIG. 1 for tracking a target area; and

FIG. 5 shows a schematic perspective view of yet an alternative embodiment of an apparatus according to FIG. 1 for tracking a target area.

### DETAILED DESCRIPTION OF PREFERRED EMBODIMENTS

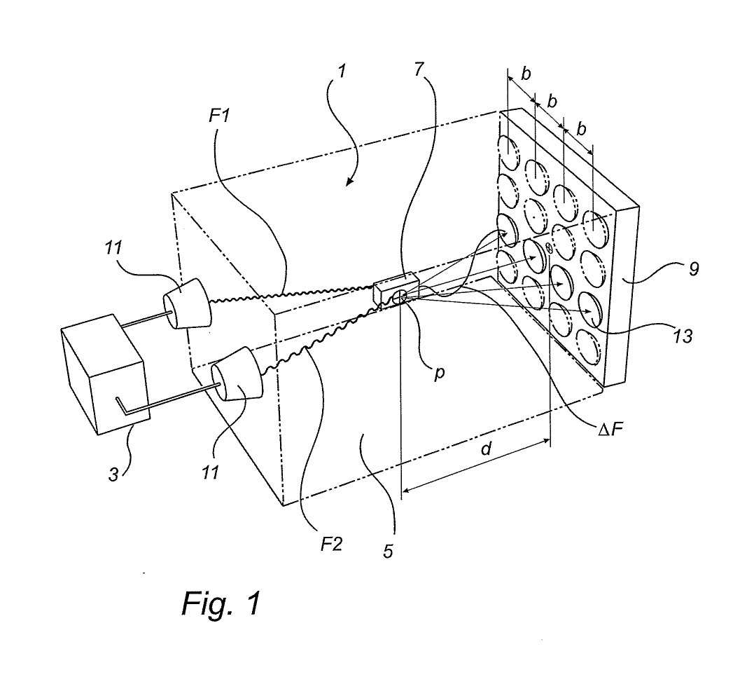

Preferred embodiments of an implant, an apparatus and a method for tracking a target area will now be described with reference to FIGS. 1-5.

The apparatus 1 according to FIG. 1 comprises an excitation source 3 located outside a lossy medium 5, a transmitter 7 located inside the lossy medium 5 and a receiver 9 located at the interface between the lossy medium 5 and the medium surrounding the lossy medium 5.

The excitation source 3 is in the illustrated embodiment according to FIG. 1 provided as two antennas 11 spaced apart from each other. It shall be noted that the excitation source 3 as well may be located within the lossy medium 5. The location of the excitation source 3 relatively to the transmitter 7 may be varied in several ways, for instance the excitation source 3 preferably may be provided in the same unit as the receiver 9. Further it shall be noted that the receiver 9 may be located within or at a distance from the lossy medium 5.

In the illustrated preferred embodiment, the excitation source 3 is arranged to emit a first energizing signal with a first frequency F1 and a second energizing signal with a second frequency F2. The first and second energizing signals have a frequency F1 and F2 in the field of microwave frequencies respectively, preferably below 3.0 GHz, especially below 1.5 GHZ and in particular between 0.5-1.5 GHz. Further, where is a slightly difference between the first frequency F1 and the second frequency F2.

Said first and second energizing signals from the excitations source is emitted in at least the direction towards the transmitter 7, for energizing of said transmitter 7. According to one preferred embodiment the transmitter 7 comprises a mixer circuit and fields storage elements (not shown). The energizing signals, with different frequencies F1 and F2, is mixed by the mixer circuit, wherein an electromagnetic signal with a differentially frequency ΔF=F1−F2 is generated by the transmitter 7. The differentially electromagnetic signal ΔF is preferably selected such as the wavelength λ of the electromagnetic signal propagating in the lossy medium 5 is greater than the intended variations of the transmitter 7 position p.

According to one embodiment the distance between the transmitter 7 and each of the sensors of the receiver 9 may be within an integer number of wavelengths λ of the electromagnetic signal propagating in the lossy medium, e.g. the distance may be between (n−1)*λ and n*λ.

In an especially preferred embodiment, the wavelength λ of the emitted signal from the transmitter 7 is greater than the distance d between the transmitter 7 and the receiver 9.

Further, the apparatus 1 according to the invention preferably is used for measuring variations from a predetermined nominal position of the transmitter 7 relatively to the receiver 9, in order to obtain a real time value for the position p of the transmitter 7 relatively to the receiver 9. The term “nominal position” could refer to a measured phase difference during an optional calibration of the system, corresponding to a pre-determined position of the transmitter 7 relatively to the receiver 9.

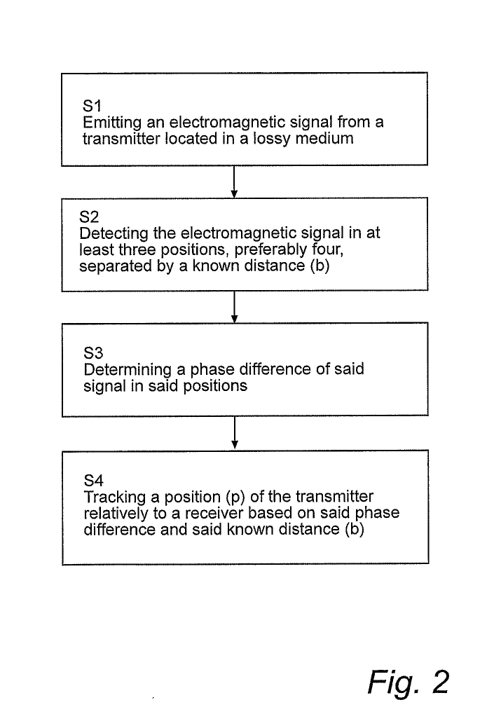

According to the first step illustrated in FIG. 2, the emitted electromagnetic differentially signal ΔF from the transmitter 7 propagates at least in the direction towards the receiver 9. The receiver 9 according to the embodiment shown in FIG. 1 is arranged with a plurality of sensors 13 for measurement of the phase difference of the received signal from the transmitter 7. In a preferred embodiment the receiver 9 is arranged with at least four sensors 13 spaced apart from each other. A distance b between each one of the adjacent sensors 13 is predetermined.

When the electromagnetic differentially signal ΔF is detected and received by the sensors 13, in accordance with the second step illustrated in FIG. 2, the phase difference is determined in accordance with the third step illustrated in FIG. 2.

According to a preferred embodiment, the phase difference is determined by using one of the sensors 13 as a reference. The signal received at one of the sensors 13 used as a reference is feed to the other sensors 13, wherein a phase difference between the signal from the reference sensor 13 and the received electromagnetic signal from the transmitter 7 is determined for each one of the sensors 13. Thereby, if one sensor 13 is used as the reference sensor to the other sensors, their measured and combined data may be used for generation of a three dimensional hyperboloid. By using all the sensors as reference sensors, more hyperboloids are generated, wherein an intersection between said hyperboloids constitute the position p of the transmitter 7 relatively to the receiver 9.

Based on the information regarding the phase difference and the known distance b between the sensors, the position p of the transmitter 7 relatively to the receiver 9 is monitored in accordance with the fourth step illustrated in FIG. 2. The position p of the transmitter 7 relatively to the receiver is calculated by the use of three dimensional geometric algorithms.

It shall be noted that the known distance b between the sensors do not have to be the same between each of the sensors. Hence, the position p of the transmitter may be determined if the sensors are arranged in a known geometry.

By measuring the electromagnetic signal emitted from the transmitter 7 in the way described above, tracking of the position of the transmitter 7 relatively to the receiver 9 may be executed in real time.

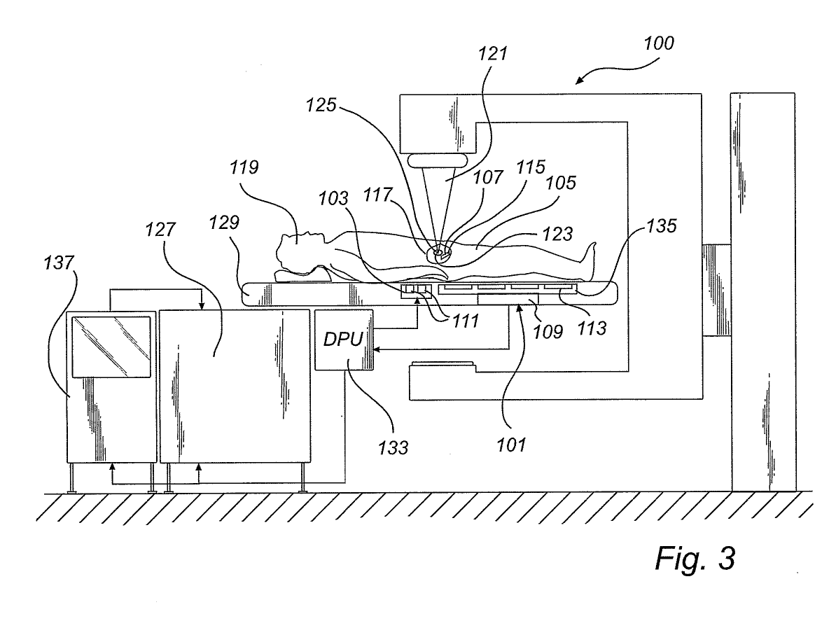

A radiotherapy treatment arrangement 100 is shown in FIG. 3 and comprises an apparatus 101, according to the invention, intended for positioning, tracking, monitoring and evaluation of an implant 115 fixable relative to a target area 117 within the living body of a patient 119.

According to one especially preferred embodiment of the invention a transmitter 107 is provided as a part of said implant 115. Further, the implant 115 is intended to be located in a living body 105 in relation to a target area 117.

In the embodiment shown in FIG. 3, the coordinates of the implant 115 position are supplied to a radiotherapy treatment unit 100, in order to obtain a treatment process with sufficient precision.

The position of the implant is determined by determination of the transmitter position. A three dimensional position of the implant may be determined as spherical coordinates, wherein the angle of the implant may be measured by combination of phase and amplitude measurements.

Further, the radiotherapy treatment arrangement 100 present in FIG. 3, comprises two iso-centers, one target area iso-center 123 and one treatment unit iso-center 125. In order to achieve a convenient accuracy for the treatment, said iso-centers 123 and 125 should coincide when ionized radiation is delivered from the radiotherapy treatment unit 100 towards the target area 117.

The apparatus 101 shown in FIG. 3 is used for tracking of the target area iso-center 123 by tracking the implant 115 position, evaluation of the coordinates differences between the target area iso-center 123 and the treatment unit iso-center 125, and, by use of the information regarding the coordinate differences, give instructions to the table movement unit 127. Said table movement unit 127 is arranged to move the table 129 in order to make the both iso-centers 123 and 125 essentially three-dimensional coincident.

Thus, the apparatus 101 is used as a guide for the radiotherapy treatment unit 100. During the treatment the apparatus 101 may be used for real time monitoring of the target area 117 position fluctuations due to the organs and/or patient 119 movements, in order to insure accurate delivery of radiation to the target area iso-center 123. Said movement of the organs may be caused by breathing, organs filling or emptying, blood flow, gastric motions etc.

A person skilled in the art understands that the apparatus 101 may be used also as a guide in other applications, where tracking and monitoring of target areas within the body is desired.

The patient 119 is provided with at least one implant 115, which is located in or adjacent to the target area 117. If more than one implant 115 is used, the position of the implants, relatively to the target area 117, is predetermined in such a way that their coordinates changes during the treatments with essentially the same proportion, which is referred to as correlated points.

Alternative, more than one implant 115 may be used if several target areas 117 shall be treated, for instance more than one tumor, wherein the implants 115 do not have to be correlated to each other.

The position of the implant 115 relative to the target area 117 is preferably predetermined. The target area iso-center 123 is chosen based on imaging data taken with imaging systems, such as CT-scan, MRI, Ultrasound, PET or the like. Based on the information from the imaging system the position for placement of the implant 115 is selected. The implant 115 is then implanted at the selected position, for instance by means of a catheter. Alternative, the positioning of the implant 115 can be based on information from other procedures, such as marking, palpation, biopsy, operation etc., wherein the information from said imaging data is not needed.

After the implant 115 is implanted in the body at the predetermined position, another imaging scanning may be executed in order to achieve the coordinates of the implant position relatively to the target area 117 to be treated. Thus, the implant 115 serves as a reference for the target area 117 position. Further, a calibration of the system for the use in one situation (e.g. radiation treatment) may be executed in relation to the imaging scanning, wherein the phase difference is measured in accordance with the abovementioned. The results from the calibration serves as a reference for further monitoring of variations of the implant 115 coordinates.

According to one preferred embodiment, the implant 115 may be provided wireless, wherein the implant 115 preferably is excited and energized by an external excitation source 103. These excitation source 103 comprises, according to one preferred embodiment, RF/microwave antennas 111, which operates at two slightly different frequencies, F1 and F2, generated by a transmitter unit. The implant 115, according to one preferred embodiment present in FIG. 3, comprises a mixer circuit and fields storage elements (not shown). Additionally, the mixer circuit is arranged to receive and mix said two RF/microwave frequencies, F1 and F2, and release a signal with an differentially frequency ΔF. The storage elements serve as loads for the implant 115 and as energy sources for the signal generated by the implant 115 by means of the mixer circuit.

According to one embodiment the emitted electromagnetic signal generated from the implant 107 may be between 5-1000 MHz, preferably 5-900 MHz and in particular 5-450 MHz.

According to an alternative embodiment, the emitted electromagnetic signal generated from the implant 107 may be between 5-350 MHz, preferably between 5-200 MHz and in particular between 5-100 MHz. Further, the wavelength of the emitted signal in the tissues of the living body is given as:

$$λ = \frac{{λ}_{0}}{\sqrt{ɛ}} = \frac{VF * {c}_{0}}{f * \sqrt{ɛ}}$$

where λ₀ is the wavelength in free space, f is the frequency, VF is the velocity factor, c₀ is wave propagation velocity in free space and ε is the permittivity of the lossy medium.

Preferably, the wave length λ of the electromagnetic signal propagating in the living body is between 0.2-2 meter, more preferably 0.4-1.5 meter and in particular 0.6-1 meter.

The signals generated by the implant 115 are detected by an array 135 provided with sensors 113 located exterior of the patient's 119 body. The array of sensors 113 are designed to measure the phase difference of the incoming from the implant 115 signals. The location of the sensors 113 follows a predefined geometry, which enabling the array 135 to serve as a fixed reference coordinate system from which the implant 115 position is calculated. The sensors 113 are connected to a receiver 109 which is designed to supply a data processing unit (DPU) 133 with information for further processing. The data processing unit 133 determines the real time implant 115 position within the body relative to the array 135 by means of incorporated algorithms. The measured phase differences using the sensors 113 are converted by a geometric 3D position calculation method, such as triangulation or neural networks etc.

Since the target area 117 is located at a predetermined position regarding the already located implant 115, the target area 117 position is calculated by the data processing unit 133 using the implant 115 position information. Further, the target area 117 position is compared with the position of the treatment unit iso-center 125.

The positions of said both iso-centers 123 and 125 are given relative to the array 135, which may serve as the fixed reference coordinate system for the calculation. If the target area iso-center 123 and the treatment unit iso-center 125 are misaligned, such that they are not three-dimensionally coincident with each other, the data processing unit 133 supplies the table moveable unit 127 with the specific instructions for table 129 readjustment. Thus, the table 129, on which the patient 119 containing the target area iso-center 123 is lying, changes its position in such a way that the two iso-centers 123 and 125 being substantially three-dimensional coincident. When the iso-centers 123 and 125 substantially coincident condition is fulfilled, the treatment unit 100 may be started for delivering of the ionized radiation towards the target area 117. The coincident criteria can be monitored in real time, which insuring the required treatment accuracy during the treatment process.

According to one embodiment the apparatus includes also a monitoring assembly 137, which is arranged to provide the data from the data processing unit 133 to a user interface, for instance accessible by a doctor or a technician operating the system. The monitoring assembly 137 may contain an opportunity of switching between manual or automatic control of the table 129 adjustment.

There may be defined a limit for the distance at which the implant 115 can be located relatively to the target area 117, keeping them properly correlated. This predetermined limit will vary for different tissues. If the implant 115 is located beyond said limit, the coordinates fluctuations of the target area 117 may not be properly described by the implant 115 coordinates fluctuations. This selected limit is referred to as the Implant Position Criteria. Generally, in order to fulfill the Implant Position Criteria, the implant 115 preferably should be located in the same tissue as the target area 117 for which the position shall be determined. For instance, in the case of the prostate, the Implant Position Criteria may be fulfilled if the implant 115 is located within the prostate.

Because of possible organ motions during the treatment, due to different factors described above, there may be defined a time interval at which the implant 115 should be positioned. The choice of this time interval depends on the different organs and tissues. Thus, in order to enhance the treatment process by increasing its accuracy, this time interval may be defined for different kind of organs and tissues. Said time interval is referred to as the Implant Time Interval Criteria.

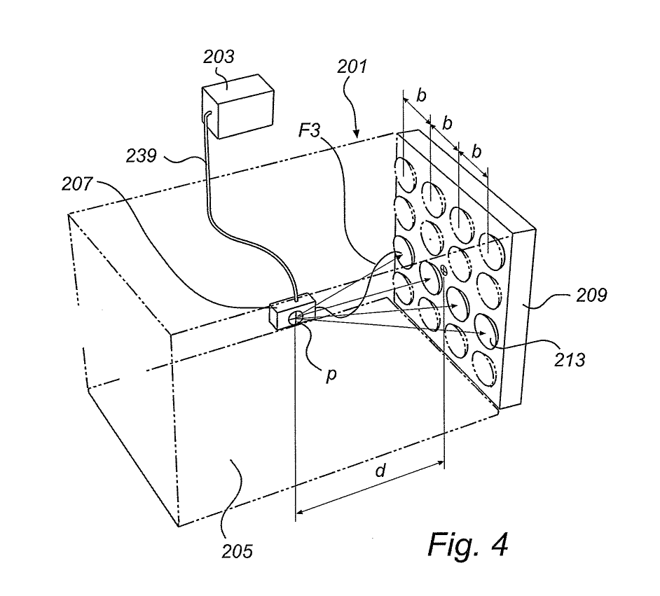

In an alternative embodiment of an apparatus 201 according to the invention, shown in FIG. 4, the transmitter 207 may be connected via a wire 239 to a excitation source 203, to allow the transmitter 207 generating the electromagnetic signal F3. For this embodiment the transmitter 207 signal is used as a reference for the later phase difference measurement with sensors 213.

For the embodiment comprising a transmitter 207 connected via a wire 239 to the excitation source 203, the sensor array measuring the phase differences by means of phase detectors. The phase difference is measured between the emitted signal from the transmitter 207, which is used as a reference, and compared with the signal detected at each sensor 213 of the array interface. Based on this information regarding the phase difference, a specific position of the transmitter 207 relatively to the array can be determined in real time. Thereby, an array comprising three sensors, or more, is sufficient to enable measurements of the transmitter position in three dimensions. Additional, for the embodiment comprising a transmitter connected via a wire, the monitoring of the transmitter position may include phase measurements, wherein the distance between the transmitter and the sensors of the receiver is determined.

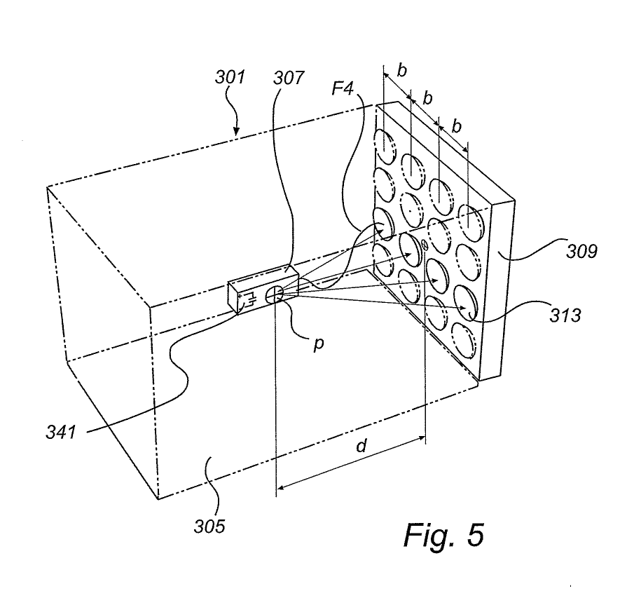

For additionally one alternative embodiment of an apparatus 301 shown in FIG. 5, the transmitter 307 may be energized by an internal source of energy 341, to allow a transmitter 307 to generate a electromagnetic signal F4. For this alternative embodiment the emitted signal F4 from the transmitter 307 is measured in a similar way as for the transmitter energized by an external source, which is described above.

It shall be noted that F3 and F4 may be selected in the same range of frequencies' as the abovementioned differentially frequency ΔF.

In a preferred embodiment, two successive pulses with different frequencies can be emitted from the transmitter. By detecting and comparing the two different pulses, reflection and attenuation within the tissues and organs of the living body may be determined.

It shall be noted that different embodiments of the present transmitters may be used as part of alternative embodiments of an implant according to the invention comprising a transmitter.

The invention shall not be interpret to only include the above described embodiments, but also combinations of the different embodiments forming alternative embodiments.

1-30. (canceled)

31. Implant fixable relative to a target area within a living body, comprising a transmitter; arranged to emit an electromagnetic signal, wherein said electromagnetic signal is adapted to propagate with a wavelength λ in said living body so that a phase difference of said electromagnetic signal in at least three positions, preferably four, separated by a known distance is detectable by a receiver for tracking variations of a position of the implant relative to said receiver, wherein said wavelength λ is selected so that:
a distance from the transmitter to each of said at least three positions, preferably four, is within one wavelength k of the electromagnetic signal, and the transmitter and the receiver operates in a near field region.

32. Implant according to claim 31, wherein said transmitter is arranged to emit an electromagnetic signal adapted to propagate with a frequency within the range of 5-1000 MHz, preferably 5-900 MHz, more preferably 5-450 MHz and especially 5-350 MHz.

33. Implant according to any one of claims 31-32, wherein said receiver comprises an array of at least four sensors, and each sensor is balanced with respect to ground.

34. Implant according to claim 33, wherein the sensors are arranged as electrostatically shielded antennas.

35. Implant according to any one of claims 31-34, wherein the transmitter is arranged to be energized by an external excitation source located outside the living body.

36. Implant according to any one of claims 31-35, wherein the transmitter comprises a frequency converter.

37. Implant according to any of claims 31-36, wherein the implant comprises a mixer circuit for receiving and mixing a first energizing signal with a first frequency and a second energizing signal with a second frequency for generation of said emitted electromagnetic signal, the frequency of said emitted electromagnetic signal substantially corresponding to the difference between said first and second frequencies.

38. Implant according to any of claims 31-36, wherein the transmitter is arranged to be energized via a wire.

39. Implant according to any one of claims 31-34, wherein the implant includes a source of energy for energizing of the transmitter.

40. Apparatus for tracking a position of a transmitter located in a lossy medium said transmitter being adapted to emit an electromagnetic signal that propagates in the lossy medium, comprising a receiver for detecting and measuring a phase difference of said electromagnetic signal in at least three positions, preferably four, separated by a known distance, for tracking the position of the transmitter relative to said receiver, wherein the at least three positions are located so that a distance from the transmitter to each one of said at least three positions is within one wavelength λ of the electromagnetic signal propagating in the lossy medium, wherein the transmitter and the receiver operates in a near field region.

41. Apparatus according to claim 40, wherein the receiver is arranged to detect and measure said electromagnetic signal emitted from the transmitter with a frequency within the range of 5-1000 MHz, preferably 5-900 MHz, more preferably 5-450 MHz and especially 5-350 MHz.

42. Apparatus according to any one of claims 40-41, wherein one of said at least three positions for detecting and measuring a phase difference of said electromagnetic signal is arranged as a reference, wherein a phase difference, for the at each position detected electromagnetic signal from the transmitter, is determined between the reference and each one of the other positions.

43. Apparatus according to any one of claims 40-42, further comprising an excitation source to be located outside the lossy medium, for energizing said transmitter.

44. Apparatus according to claim 43, wherein said excitation source is arranged to emit a first energizing signal with a first frequency and a second energizing signal with a second frequency, for generation of said emitted electromagnetic signal, the frequency of said emitted electromagnetic signal substantially corresponding to the difference between said first and second frequencies.

45. Apparatus according to claim 44, wherein the excitation source comprises two antennas arranged for emitting said first and second energizing signals respectively.

46. Apparatus according to any one of claims 40-45, wherein the receiver comprises an array of at least four sensors with a known distance between adjacent sensors.

47. Apparatus according to claim 46, wherein each sensor is balanced with respect to ground.

48. Apparatus according to claim 47, wherein the sensors are arranged as electrostatically shielded antennas.

49. Apparatus according to any one of claims 40-48, wherein said distance is shorter than half a wavelength λ of the detected electromagnetic signal propagating in the lossy medium.

50. System for tracking a target area in a lossy medium, comprising an apparatus according to any of the claims 40-49, and a transmitter fixable relative to said target area and adapted to emit a signal propagating in the lossy medium.

51. System according to claim 40, wherein the transmitter is provided as a part of an implant (115) according to any one of the claims 31-39.

52. Method for tracking a position of a transmitter located in a lossy medium, comprising
emitting an electromagnetic signal from the transmitter,
detecting said electromagnetic signal in at least three positions, preferably four, located so that a distance from the transmitter to each one of said at least three positions is within one wavelength λ of the electromagnetic signal propagating in the lossy medium, wherein said at least three positions are separated by a known distance,
determining a phase difference of said signal in said positions, and
tracking the position of the transmitter relative to a receiver based on said phase difference and said known distance, and
selecting wavelength λ of the electromagnetic signal so that the transmitter and the receiver operates in a near filed region.

53. Method according to claim 52, wherein a frequency of the emitted electromagnetic signal from the transmitter is within the range of 5-1000 MHz, preferably 5-900 MHz, more preferably 5-450 MHz and especially 5-350 MHz.

54. Method according to any one of claims 52-53, wherein said receiver is selected to be an array of at least four sensors, and each sensor being balanced with respect to ground.

55. Method according to any claim 54, wherein the sensors are selected to be electrostatically shielded antennas.

56. Method according to any one of claims 52-55, further comprising energizing the transmitter by means of an excitation source located outside the lossy medium, for generation of said electromagnetic signal.

57. Method according to claim 56, wherein the step of energizing the transmitter further comprises generating at least one energizing signal emitted from the excitation source, wherein the step of emitting a signal from the transmitter further comprises:
converting said energizing signal received by the transmitter from the excitation source, and
generating said emitted electromagnetic signal.

58. Method according to claim 56, wherein the step of energizing the transmitter further comprises generating a first energizing signal with a first frequency and a second energizing signal with a second frequency, emitted from the excitation source, and wherein the step of emitting a signal from the transmitter further comprises:
mixing said first and second energizing signals received by the transmitter from the excitation source, and
generating said emitted electromagnetic signal, wherein the frequency of said emitted electromagnetic signal substantially corresponding to the difference between said first and second frequencies.

59. Method according to any one of claims 52-58, wherein the transmitter is located in a living body.

60. Use of the method according to any one of claims 52-59, for treatment of a target area in a living body by means of a radiation therapy treatment arrangement.
